(19) United States
(12) Patent Application Publication
(10) Pub. No.: US 2023/0338414 A1
(43) Pub. Date: Oct. 26, 2023

(54) USE OF TLR4 MODULATOR IN THE TREATMENT OF COCCIDIOSIS

(71) Applicant: ZIVO Bioscience, Inc., Bloomfield Hills, MI (US)

(72) Inventors: Andrew A. Dahl, Bloomfield Hills, MI (US); William P. Pfund, Bloomfield Hills, MI (US); Amy E. Steffek, Bloomfield Hills, MI (US)

(73) Assignee: ZIVO Bioscience, Inc., Bloomfield Hills, MI (US)

(21) Appl. No.: 18/139,749

(22) Filed: Apr. 26, 2023

Related U.S. Application Data
(63) Continuation-in-part of application No. 17/320,706, filed on May 14, 2021, now abandoned.

(51) Int. Cl. A61K 31/739; A61K 35/74; A61K 36/02; A61P 33/02; A23K 20/163; A23K 10/16; A23K 50/75

(52) U.S. Cl. CPC A61K 31/739; A61K 35/74; A61K 36/02; A61P 33/02; A23K 20/163; A23K 10/16; A23K 50/75

(57) ABSTRACT

An effective treatment mechanism in controlling a variety of diseases by modulating the inflammatory response often associated with disease is disclosed. The disclosed inventive concept is based on the modulation of TLR4 by use of a member of the Variovorax group or the Rhodobacter group. Specifically, the Gram-negative bacterium Variovorax paradoxus or the Gram-negative bacterium Rhodobacter sphaeroides is used according to the disclosed inventive concept in the treatment of disease by reducing or inhibiting inflammatory responses.

### CROSS REFERENCE TO RELATED APPLICATION

This continuation-in-part application claims the benefit of U.S. Ser. No. 17/320,706 entitled “Use of TLR4 Inhibitor in the Treatment of Coccidiosis” filed May 14, 2021, which is a US. Non-provisional patent application of U.S. Provisional Patent Application No. 63/024,886, entitled “Use of TLR4 Inhibitor in the Treatment of Coccidiosis,” filed May 14, 2020, which are herein incorporated by reference in their entireties for all purposes.

According to one exemplary embodiment, a composition is derived from the culture or co-culture of specific bacteria of ATCC Item No. SD-8636 received by the ATCC on or about Aug. 25, 2022.

In various embodiments, a method of treating coccidiosis in animals comprises administering to the animal a therapeutically effective amount of biomass from the culture or co-culture of specific bacteria of ATCC Item No. SD-8636 received by the ATCC on or about Aug. 25, 2022.

In various embodiments, a composition for the modulation of the TLR pathway in animals comprising a lipopolysaccharide derived from Gram-negative bacteria of ATCC Item No. SD-8636 received by the ATCC on or about Aug. 25, 2022.

### TECHNICAL FIELD

The present invention relates to Toll-like Receptor 4 (TLR4) and its modulation in the treatment of disease. More particularly, the present invention relates to the use of a lipopolysaccharide (LPS) from a Gram-negative bacteria in the selective modulation of TLR4.

### BACKGROUND OF THE INVENTION

Lipopolysaccharide (LPS) is a component found in the outer membrane of many Gram-negative bacteria. Toll-like Receptor 4 (TLR4) is a protein that is a member of a family of toll-like receptors. TLR4 recognizes LPS and may be stimulated to mediate the production and release of pro-inflammatory cytokines which leads to the activation of the innate immune system.

It may be desirable under certain circumstances to selectively modulate the TLR4 cascade either by directly activating or inhibiting the TLR4 molecule itself or at any other point downstream in the associated pathway. Regardless of the point of inhibition, the result is the slowing or halting of the production of pro-inflammatory cytokines, thereby improving immune health under certain circumstances. Inhibition is achieved by blocking the signaling needed to mediate the production and release of the cytokines. In other circumstances, selectively activating TLR4 and the associated downstream pathway may lead to an heightened or accelerated immune response to an invading pathogen thereby enhancing an animals ability to prevent or combat disease.

The modulation of pro-inflammatory cytokines is an important factor in the treatment and prevention of certain diseases in humans and many animal species. A non-limiting example of such a disease is coccidiosis, a common and extremely destructive disease in the poultry industry. The disease leads to damage of the intestinal system of the host which often predisposes animals to other detrimental conditions such as necrotic enteritis and, ultimately, may lead to death of the animal. The host's inflammatory response to the disease contributes to the intestinal damage and susceptibility to infection by other pathogens such as Clostridium perfringens, the causative agent for necrotic enteritis.

The current treatment regimen for diseases such as coccidiosis includes the use of antibiotics, ionophores, or other chemical agents. However, while providing a degree of success, these treatments add a considerable cost to the poultry industry. In addition, the overuse of antibiotics in the poultry industry raises concerns about an increase in resistance to one or more antibiotics. Accordingly, it is desirable to develop a non-antibiotic based treatment of pathogenic infections such as coccidiosis in poultry.

### SUMMARY OF THE INVENTION

The disclosed inventive concept provides an effective treatment for a broad variety of diseases through the modulation of the inflammatory response normally associated with the disease. A non-limiting exemplary use of the disclosed inventive concept as a treatment for disease is its use as a replacement for coccidiostatsin the treatment of parasitic infections such as coccidiosis. The modulated inflammatory response has been found to result in improved intestinal morphology including the promotion of intestinal barrier integrity. The improvement in poultry health was achieved without the use of antibiotics. Delivery of the composition is made by oral administration of the active materials mixed into feed or drinking water.

The disclosed inventive concept is based on the modulation of the TLR pathway by a compound produced from a Gram-negative bacterial strain such as a member of the Variovorax group or a member of the Rhodobacter group. Specifically, the Gram-negative bacterium Variovorax paradoxus or the Gram-negative bacterium Rhodobacter sphaeroides may be used in disease treatment according to the disclosed inventive concept by modulating inflammatory responses.

Accordingly, the disclosed inventive concept is set forth as a compound capable of selectively modulating the TLR4 signaling pathway. The compound comprises a lipopolysaccharide derived from a member of the Variovorax group or the Rhodobacter group.

In a preferred embodiment, the lipopolysaccharide is derived from the Gram-negative bacterium Variovorax paradoxus or the bacterium Rhodobacter sphaeroides.

In another preferred embodiment, the lipopolysaccharide compound derived from one of Variovorax paradoxus or Rhodobacter sphaeroides or from both is incorporated within an grain-based feed to improve the gut health of poultry.

### DESCRIPTION OF THE DRAWINGS

For a more complete understanding of this invention, reference should now be made to the accompanying figures. As set forth in the figures, the designation “No Tx, No Challenge” refers to a test in which no treatment was administered to a subject animal not deliberately infected with coccidiosis. The designation “No Tx, Cocci” refers to a test in which no treatment was administered to a subject animal deliberately infected with coccidiosis. The designation “Anti-cocci, Cocci” refers to a test in which the subject animal was infected with coccidiosis and the animal was administered an anticoccidial.

The designation “ZIVO A, Cocci” refers to a test in which the subject animal was infected with coccidiosis and the animal was administered a first treatment composition according to the disclosed inventive concept. The designation “ZIVO S, Cocci” refers to a test in which the subject animal was infected with coccidiosis and the animal was administered a second treatment composition according to the disclosed inventive concept. The designation “ZIVO T-hi, Cocci” refers to a test in which the subject animal was infected with coccidiosis and the animal was administered a third treatment composition according to the disclosed inventive concept. The designation “ZIVO T-low, Cocci” refers to a test in which the subject animal was infected with coccidiosis and the animal was administered a fourth treatment composition according to the disclosed inventive concept.

The accompanying figures are described as follows:

FIG. 1 is a graph illustrating test subject feed conversion data for Days 0 to 7;

FIG. 2 is a graph illustrating test subject feed conversion data for Days 0 to 14.

FIG. 3 is a graph illustrating test subject feed conversion data for Days 0 to 21;

FIG. 4 is a graph illustrating test subject feed conversion data for Days 0 to 28;

FIG. 5 is a graph illustrating test subject feed conversion data for Days 0 to 42;

FIG. 6 is a graph illustrating test subject mortality for Days 0 to 7;

FIG. 7 is a graph illustrating test subject mortality for Days 0 to 14;

FIG. 8 is a graph illustrating test subject mortality for Days 0 to 21;

FIG. 9 is a graph illustrating test subject mortality for Days 0 to 28;

FIG. 10 is a graph illustrating test subject mortality for Days 0 to 42;

FIG. 11 is a graph illustrating test subject lesion scores determined on Day 21;

FIG. 12 is a graph illustrating test subject lesion scores determined on Day 42;

FIG. 13 is a graph illustrating test subject duodenum loop oocycst count (gram/bird/area) on Day 21;

FIG. 14 is a graph illustrating test subject duodenum loop oocycst count (gram/bird/area) on Day 42;

FIG. 15 is a graph illustrating test subject mid-gut oocycst count (gram/bird/area) on Day 21;

FIG. 16 is a graph illustrating test subject mid-gut oocycst count (gram/bird/area) on Day 42;

FIG. 17 is a graph illustrating test subject whole cecum oocycst count (gram/bird/area) on Day 21;

FIG. 18 is a graph illustrating test subject whole cecum oocycst count (gram/bird/area) on Day 42;

FIG. 19 is a graph illustrating test subject Campylobacter fecal count on Day 21;

FIG. 20 is a graph illustrating test subject Campylobacter fecal count on Day 42;

FIG. 21 is a graph illustrating test subject Campylobacter cecum count on Day 21;

FIG. 22 is a graph illustrating test subject Campylobacter cecum count on Day 42;

FIG. 23 is a graph illustrating test subject Salmonella fecal count on Day 21;

FIG. 24 is a graph illustrating test subject Salmonella fecal count on Day 42.

FIG. 25 is a graph illustrating test subject Salmonella cecum count on Day 21;

FIG. 26 is a graph illustrating test subject Salmonella cecum count on Day 42;

FIG. 27 is a graph illustrating test subject Clostridium perfringens fecal count on Day 21;

FIG. 28 is a graph illustrating test subject Clostridium perfringens fecal count on Day 42;

FIG. 29 is a graph illustrating test subject E. coli fecal count on Day 21;

FIG. 30 is a graph illustrating test subject E. coli fecal count on Day 42;

FIG. 31 is a graph illustrating test subject feed consumption on Days 0-42;

FIG. 32 is a graph illustrating test subject average body weight in grams on Days 0-42; and

FIG. 33 is a graph illustrating test subject average weight gain in grams per day on Days 0-42.

### DETAILED DESCRIPTION OF THE PREFERRED EMBODIMENT

In the following description, various operating parameters and components are described for different constructed embodiments. These specific parameters and components are included as examples and are not meant to be limiting. Unless otherwise noted, all technical and scientific terms used herein are to be accorded their common meanings as would be understood by one having ordinary skill in the art.

The Compounds Used in Treatment

In general, delivery of the composition is made by oral administration of the active materials mixed into feed or drinking water. The disclosed method of treatment preferably, but not absolutely, utilizes a compound generally derived from a lipopolysaccharide (LPS) of Gram-negative bacteria. By administering the compound early in broiler life, disease prevention and treatment via immune modulation are achieved. As used herein, the term “inhibitor” refers to a molecule that reduces or attenuates the activity induced by another molecule, receptor, cellular structure, or organ. By way of example, a compound that might block the LPS-dependent activation of TLRs, such as but not limited to TLR4, present on the surface of a host immune cell would be regarded as an inhibitor of this particular pathway. Conversely, the term “activator” or “agonist” refers to a molecule that increases or enhances the activity induced by another molecule, receptor, cellular structure, or organ.

As used herein, the term “algal culture” is defined as an algal organism and bacteria (one or more types) that grow together in a liquid medium. Unless expressly stated otherwise, the term “algal biomass” refers to the algal cells and bacterial cells (with the liquid culture medium removed). The “algal biomass” can be wet material or dried material.

Unless expressly stated otherwise, the term “algal supernatant” is defined as the culture medium in which the algal biomass is grown that contains excreted compounds from the algal biomass. Algal supernatant is obtained by growing algal biomass in culture medium for an appropriate length of time and then removing the algal and bacterial cells by filtration and/or centrifugation.

It is known that bacteria of the Variovorax genus and the Rhodobacter genus are metabolically versatile. Variovorax is a Gram-negative aerobic bacterium that can grow under a variety of conditions. It is part of the subclass Proteobacteria and is capable of metabolically utilizing several natural compounds generated by plants or algae. Rhodobacter can grow under a broad variety of conditions, utilizing both photosynthesis and chemosynthesis. Growth can also be achieved under both anaerobic and aerobic conditions. Rhodobacter sphaeroides represents a Gram-negative facultative bacterium and is a member of the α-3 subdivision of the Proteobacteria.

Embodiments of the compound used in the treatment of disease as set forth herein include one or more LPS/Lipid A compounds produced by Gram-negative bacterial strains for use as selective modulators of the TLR signaling pathway, such as the TLR4 pathway. The disclosed inventive concept involves any combination of three fundamental steps: (1) the Gram-negative bacteria produces LPS/Lipid A compounds; (2) the LPS/Lipid compounds modulate TLR4 activity through inhibition or activation; and (3) a downstream effect results in modulated inflammation and recruitment of immune cells of the gut via the modulation of TLR4 signaling, thereby aiding in the treatment of coccidiosis, necrotic enteritis, and other conditions related to gut inflammation.

In an embodiment, the LPS/Lipid A compounds used as selective modulators of the TLR4 signaling pathway are produced from a Variovorax paradoxus strain. The Variovorax paradoxus strain may be a naturally occurring strain.

In another embodiment, the LPS/Lipid A compounds used as selective modulators of the TLR4 signaling pathway are produced from a Rhodobacter sphaeroides strain. Extensive studies have been undertaken regarding the structure and function of Rhodobacter sphaeroides. More focused studies have examined the photosynthetic characteristics of Rhodobacter sphaeroides. It is known that lipopolysaccharides from Rhodobacter sphaeroides are effective TLR4 antagonists in human cells that prevent TLR4-mediated inflammation by blocking LPS/TLR4 signaling. In cells of other species, LPS from Rhodobacter sphaeroides acts as an agonist of the TLR4 pathway. The inventors employed a testing methodology to address multiple immune response mechanisms in poultry to arrive at the conclusion that an LPS compound derived from Rhodobacter sphaeroides proved effective as a coccidiostat in poultry. Initial data suggested modulation by an LPS-like molecule, it was not until specific testing directed to Rhodobacter sphaeroides revealed the effectiveness of this bacterium in the treatment of disease, such as in the treatment of coccidiosis in poultry. Research further showed that combining a TLR4 inhibitor with an activator of TLR2 (such as lipoprotein from Gram-negative bacteria) provides an anti-coccidiosis effect.

Accordingly, embodiments of the compound used in the treatment of disease according to the present disclosure are directed to one or more LPS/Lipid A compounds produced by a Gram-negative bacterial strain of the group Variovorax or the group Rhodobacter for use as selective modulators of the TLR4 signaling pathway. A specific embodiment of the disclosed inventive concept is directed to the use of an LPS/Lipid A compound used as a selective modulator of the TLR4 signaling pathway produced from the Variovorax paradoxus strain and the Rhodobacter sphaeroides strain.

The LPS/Lipid A compound employed herein may be obtained from the Variovorax paradoxus strain and/or the Rhodobacter sphaeroides strain by any suitable method, but in specific embodiments they are extracted using standard multi-step LPS extraction protocols, such as: (1) extracting freeze-dried bacteria with a solution of phenol/guanidine thiocyanate and collecting the water layer for freeze-drying; (2) resolubilizing the freeze-dried fraction in water; (3) ultrafiltration of the solubilized fraction to remove low molecular weight substances and salts; (4) affinity purifying the high-molecular weight fraction using a polymyxin B resin column such as Affi-prep polymyxin matrix material (Bio-Rad), from which an active fraction is eluted with 1 deoxycholate and, optionally; (5) performing additional purification using size-exclusion chromatography.

In some examples, multiple types of LPS extraction protocols are employed to obtain an LPS compound from the bacteria, and extraction procedures may be performed more than once. Once the LPS compound is extracted and purified from the bacteria, the Lipid A fraction may be prepared by acid hydrolysis or other suitable technique.

The one or more LPS/Lipid A compounds derived from Gram-negative bacterial strains, such as Variovorax paradoxus or Rhodobacter sphaeroides, may selectively modulate the TLR4 signaling pathway to modulate inflammatory responses and to improve immune health in a variety of uses and applications. In an embodiment, the LPS/Lipid A compound derived from Variovorax paradoxus or Rhodobacter sphaeroides may be incorporated within an grain-based feed to improve gut health of poultry.

The disclosed LPS/Lipid A compound derived from Variovorax paradoxus or Rhodobacter sphaeroides may be used to improve the health of poultry through a variety of mechanisms. For example, if acting as an inhibitor, the LPS/Lipid A compound may protect against internal inflammation in poultry by negatively regulating inflammatory mediators via the downregulation of TLR4 expression and the downstream inhibition of NF-kappa B activation in a typical inflammatory cascade. In another example, the LPS/Lipid A compound may inhibit the activation of TLR4 in poultry by interfering with cysteine residue-mediated receptor dimerization. In yet another example, the LPS/Lipid A compound may inhibit the ability of non-infectious and infectious stimuli to interact with TLR4 and trigger a pro-inflammatory response, thereby improving poultry gut integrity. Alternatively, if working as an agonist of the TLR4 pathway, LPS/Lipid A compound may prime the immune system to better response to invading pathogens by recruiting specific disease fighting immune cells to intestinal tissues in advance of a disease challenge thereby accelerating and heightening the immune response to any subsequent pathogen exposure.

Specific Treatment Compounds

The disclosed treatment compounds are based on one or more fresh water algal biomasses including bacterial strains as discussed above. More particularly, the algal biomass may include the Gram-negative such as Variovorax paradoxus strain or Gram-negative Rhodobacter sphaeroides strain.

As noted, four treatment compounds are presented and considered. The compounds share the common characteristic of the algal biomass referenced above and are used in animal treatment. The algal biomass-based products are fed to animals in a formulated diet such as a corn or corn-soybean meal (SBM) diet or are delivered in drinking water. As noted, the specific treatment compositions include “ZIVO A,” “ZIVO S,” “ZIVO T-hi,” and “ZIVO T-low.”

ZIVO A Treatment Compound

The ZIVO A Treatment Compound is fresh water algal biomass containing Gram-negative bacteria provided as animal feed in combination of a feed additive, such as soy oil, preferably though not exclusively at a ratio of two parts soil oil to one part algal biomass. Once the biomass and feed additive are combined to the preferred premix level, the combined batch is poured or administered evenly into a ribbon mixer containing finished feed. The combined batch is preferably provided in an amount of between about 0.5 lbs. per ton and about 11.0 lbs. per ton of finished feed and is more preferably though not exclusively provided in an amount of about 3.5 lb/ton of feed with good efficacy without being wasteful. In general, treatment using ZIVO A Treatment Compound is around 700 mg per bird per a 42 day period.

ZIVO S Treatment Compound

The ZIVO S Treatment Compound is a liquid algal supernatant (representing the culture media collected following growth therein of the fresh water algae). Preferably but not absolutely the ZIVO S Treatment Compound is a 500× liquid algal supernatant diluted in drinking water for consumption by animals preferably though not absolutely in the amount of 400 mcl of the 500× stock is added to each liter of drinking water and mixed thoroughly. In general, treatment using ZIVO S Treatment Compound is around 9 g per bird per a 42 day period.

ZIVO T-hi and T-low LPS Treatment Compounds

The ZIVO T-hi and T-low LPS Treatment Compounds include both LPS-RS, representing Rhodobacter sphaeroides-derived purified lipopolysaccharide, and LPS-VP, representing Variovorax paradoxus-derived purified lipopolysaccharide. In general, treatment using ZIVO T-hi or T-low Treatment Compounds is around 20 mg per bird per a 42 day period. The ZIVO T-hi LPS Treatment Compound is provided in vials containing 5 mg of lyophilized product. Once solubilized, the product is stable for one month when stored in a refrigerator (4° C.). When needed, each vial is solubilized with the addition of one mL of endotoxin-free water and is then vortexed for 30 seconds or until complete solubilization is achieved based on visual determination. For a best outcome, the T-hi Treatment Composition is stored in a freezer (−20° C.) until needed.

For the ZIVO T-hi LPS-RS Treatment Compound, the solubilized product is added to water at a rate of 4 mcL per liter of water (i.e., 0.004 mL/L) and mixed thoroughly. For the ZIVO T-low LPS-RS Treatment Composition, “Low Dose” treatment group, the solubilized product is added to water at a rate of 0.4 mcL per liter of water (i.e., 0.0004 mL/L) and mixed thoroughly.

“Intermediate” stock solutions may be prepared to allow for more convenient transfer volumes provided that the final product concentrations of purified lipopolysaccharide in drinking water are 20 mcg/L and 2 mcg/L for the ZIVO T-hi and ZIVO T-low groups, respectively.

Studies

Studies were undertaken to determine the response and efficacy of the various treatment compounds. Pellet feed was employed for the ZIVO A Treatment Compound using a corn-soybean diet type commercial ration formulation. Two test substances were also administered in drinking water which included the ZIVO S Treatment Compound and the ZIVO T-hi and T-low LPS-RS Treatment Compounds.

Study—Treatment Method

A total of 2,184 mixed sex broiler chicks were obtained within twelve hours of hatching from fecal contaminated flocks at a commercial hatchery on Day 0 (hatch and placement day). A number of mixed-sex broiler chicks (50:50 sex ratio) were randomly assigned on Day 0 by individual weights to one of several test group pens, each with replicates. Only antibiotic-free birds were sourced, and no coccidiosis vaccine was administered at the hatchery or at any time during the study. Chicks were evaluated upon receipt for signs of disease or other complications that could affect study outcome. Weak birds were humanely sacrificed. Birds were not replaced during the study.

Following examination, chicks were weighed and allocated to pens for the various treatment groups using a randomized block design. Weight distribution across the treatment groups was assessed prior to feeding by comparing the individual test groups' standard deviations of the mean against that of the control group. Weight distribution across the groups was considered acceptable for this study when differences between control and test groups were within one standard deviation.

Treatment Groups—Treatment groups, the levels of test material, the number of replicates, the number of bird replicates, and the routes of administration were established as follows.

| | | | | Route | | |
|---|---|---|---|---|---|---|
| Trt. | | Cocci | Treatment Substance | of | | Birds per |
| No. | Treatment Description 2, 3 | Challenge | and Level | Admin | Reps | Replicate |
| 1 | Unchallenged/No Treatment | No | None | Feed | 12 | 26 |
| | Control | | | Pellets1 | | |
| 2 | Challenged/No Treatment | Yes | None | Feed | 12 | 26 |
| | Control | | | Pellets1 | | |
| 3 | Antibiotic Control | Yes | Coban, 90 g/ton of | Feed | 12 | 26 |
| | | | finished feed | Pellets1 | | |
| 4 | Algae Biomass (ZIVO A) | Yes | 3.5 lb/ton of finished | Feed | 12 | 26 |
| | | | feed | Pellets1 | | |
| 5 | Algal Culture Supernatant | Yes | 400 mcL/L of | Water | 12 | 26 |
| | (500×) (ZIVO S) | | drinking water | | | |
| 6 | LPS-RS Solution (ZIVO T-hi) | Yes | 20 mcg/L of drinking | Water | 12 | 26 |
| | | | water | | | |
| 7 | LPS-RS Solution (ZIVO T-low) | Yes | 2 mcg/L of drinking | Water | 12 | 26 |
| | | | water | | | |

1Corn and SBM rations, with normal nutritional formulations.
2 No Coccidiostat or ABF (Antibiotic Free Products) administered during the entire study. One control antibiotic and four test materials were fed to the birds.
3 No Coccidiosis-Vaccine was administered at the hatchery or during the course of this study.

All birds received nutritionally adequate food or drink compounds. Birds were fed their respective treatment diets ad libitum from day of hatch to 42 days of age, the typical average market age of broiler chickens in US. Birds were raised on built-up litter to further mimic stress conditions typically experienced in poultry production.

For the ZIVO A Treatment Compound, diets were weighed at the beginning of each formulation period and fed in three phases: Starter diet (0-21 days of age), Grower diet (22-35 days of age) and Finisher diet (36-42 days of age). Diets were fed for the entire study duration as pellets (with pellets served as crumbles on days 0-21). All treatment compound diets were offered ad libitum without restrictions to full-fed consumption, except for an 8-hour fasting period for cocci-inoculated birds prior to cocci-challenge on Day 7.

On Day 7 and 7-days of age (Trial Day 0=hatch and placement day), adequate feed was precisely weighed, provided to consume at the rate of 100% fill-capacity on average for all birds. This was be determined by measuring the quantity of feed consumed within a 24-hr period the day before for each pen. Also on Day 7 all birds in the challenged groups received oocyst-inoculated sustenance containing a mixture of Eimeria acervulina, Eimeria maxima, and Eimeria tenella. Particularly, the birds received sustenance containing a mixture 100,000 oocysts per bird of E. acervulina, 50,000 oocysts per bird of E. maxima, and 75,000 oocysts per bird of E. tenella.

Cocci-Challenge Model —. All challenge organisms were mixed in the Starter Feed using a 50# mixer with a thorough mix running time of about ten minutes. Prior to the challenge, all cocci-inoculated birds were starved for eight hours. Inoculated feed was provided to the birds. After two hours, all remaining inoculated feed was removed and weighed to assure equal consumption per pen and per bird. The quantity of feed (both placed and withdrawn) was recorded on each pen's feed record.

Throughout the study, birds were observed at least three times daily for overall health, behavior, and evidence of toxicity. Pens were monitored for environmental conditions, including temperature, lighting, water, feed, litter condition, and unanticipated house conditions/events. Pens were checked daily for mortality. Examinations were performed on all broilers found dead or moribund. Mortalities were recorded (date and weight) and examined (both internal and external body mass). Throughout the study, birds were reared on built-up litter from a minimum of three previous flocks obtained from a local chicken farm to simulate stress-induced health risks related to commercial production.

Sample Collection Schedules—The studies adhered to the following collection schedules:

| Data/Sample | | | |
|---|---|---|---|
| Collected | When | Sample Size | Measurements |
| FI, BW, and mortality | Weekly | Individual weights by | FI, BW, BWG, Adjusted |
| | | sex (7, 14, 21, 28, and | FCR, mortality, BW, |
| | | 42 days) | coefficient of variation) |
| Fecal samples for: E. | Days 21 and 42 | 4 birds/pen at 21 days | Enumeration of E. |
| acervuline in loop of | | and 10 birds/pen at 42 | acervuline in loop of |
| small intestine area, E. | | days | small intestine area, E. |
| maxima in jejunum, and | | | maxima in jejunum, and |
| E. tenella in ceca. | | | E. tenella in ceca |
| Both Gut Lesion Score | Days 21 and 42 | 4 birds/pen at 21 days | Lesion scores (both |
| and Coccidia Lesion | | and 10 birds/pen at 42 | normal gut and |
| Incidence Score of | | days | coccidian lesion |
| small intestine | | | incidence score) |
| Fecal samples for: | Days 21 and 42 | 4 birds/pen at 21 days | Emeria spp. Counts |
| Digesta from small | | and 10 birds/pen at 42 | enumerated from both |
| intestine and ceca | | days | small intestine and ceca |
| Fecal samples for: | Days 21 and 42 | 4 birds/pen at 21 days | Salmonella & |
| Digesta from small | | and 10 birds/pen at 42 | Campylobacter |
| intestine and ceca | | days | incidence; E. coli, APC, |
| | | | and C. Perfringens |
| | | | enumeration |
| Villi Cell Height, Crypt | Days 21 and 42 | 4 birds/pen at 21 days | Villi Cell Height, Crypt |
| and Villus/Crypt ratio | | and 10 birds/pen at 42 | and Villus/Crypt ratio |
| | | days | |

Study Evaluation

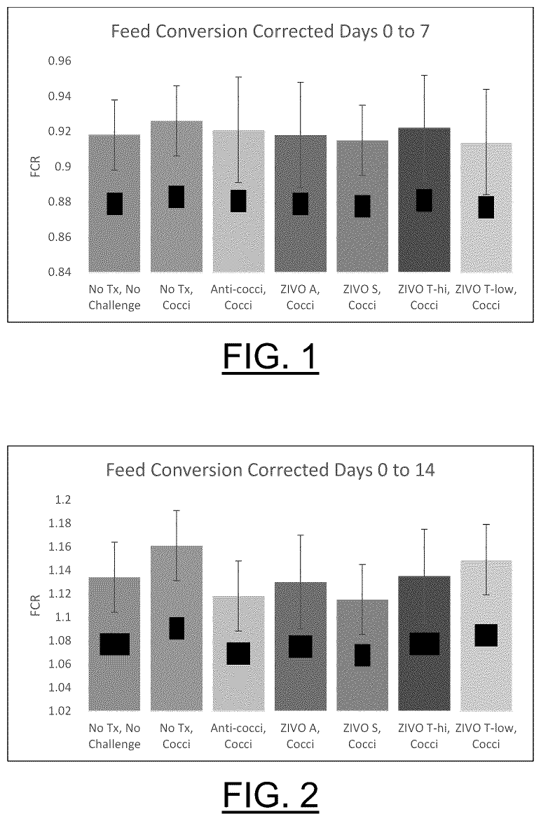

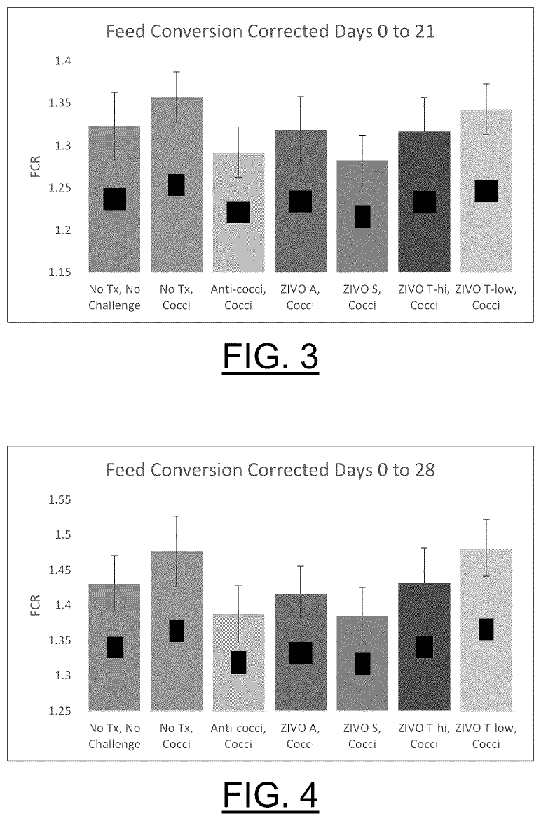

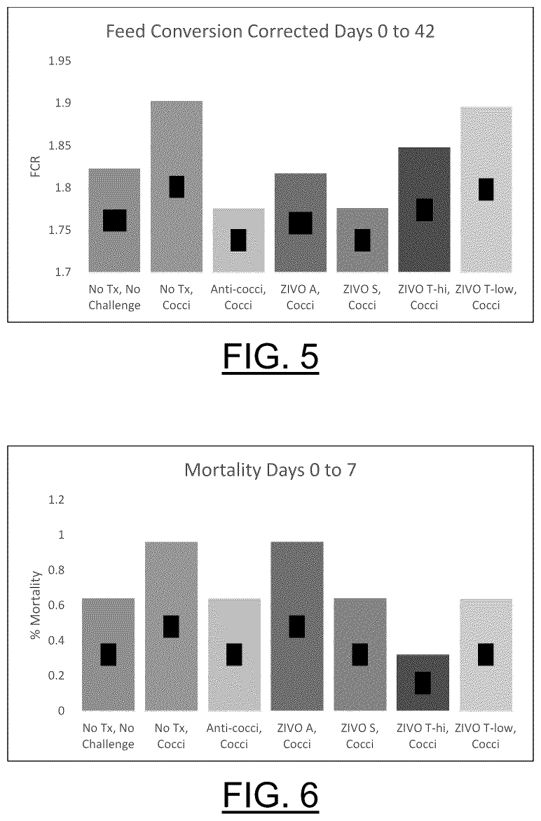

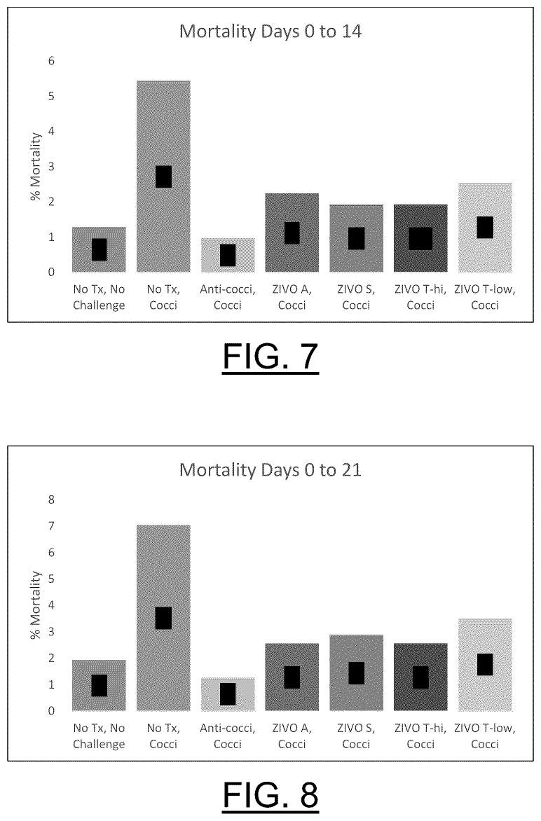

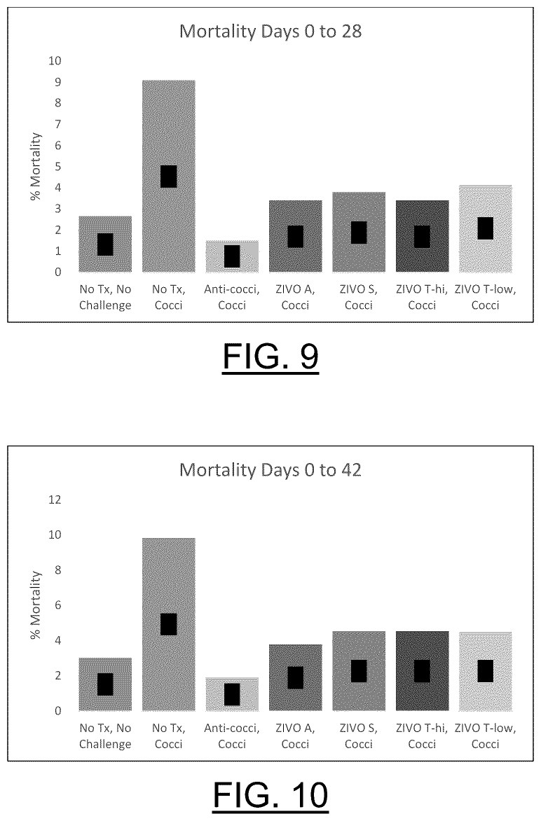

Differences between the untreated and non-diseased birds, the untreated diseased birds, the diseased birds treated with a conventional antibiotic over various periods of time between 0 and 42 days, and the diseased birds treated with different inventive compounds are illustrated in the graphs shown in FIGS. 1 through 33. The graphs are directed to feed conversion ratios (FCRs), morality, lesion scores, duodenum loop oocyst counts, mid-gut oocyst counts, whole cecum oocyst counts, various fecal counts (campylobacter, salmonella, Clostridium perfringens, and E. coli), average body weight, feed consumption rats, and average weight gain.

Feed Conversion—As illustrated in FIGS. 1 through 5, mortality-corrected Feed Conversion Ratio was measured and reported for Days 0-7, 0-14, 0-21, 0-28 and 0-42. The disclosed inventive compounds consistently provided improved results when compared with the untreated and coccidiosis-diseased group. Most notable are the positive results achieved by the application of the ZIVO T-hi Treatment Compound which demonstrates improvement over the antibiotic treated infected birds across all samplings

Mortality—As illustrated in FIGS. 6 through 10, mortality was calculated for Days 0-7, 0-14, 0-21, 0-28 and 0-42. Across all age periods, the % mortality of the untreated and diseased group was consistently higher than for the groups treated with ZIVO A, ZIVO S, ZIVO T-hi, and ZIVO T-low. At Days 0 to 7 (shown in FIG. 6) the differences in mortality rates are relatively dramatic with a notable improvement demonstrated by the ZIVO T-hi group. Over time and most vividly by Days 0 to 42 (shown in FIG. 10) all of the groups treated with ZIVO A, ZIVO S, ZIVO T-hi, and ZIVO T-low had significantly reduced mortality compared with the group of untreated and diseased birds.

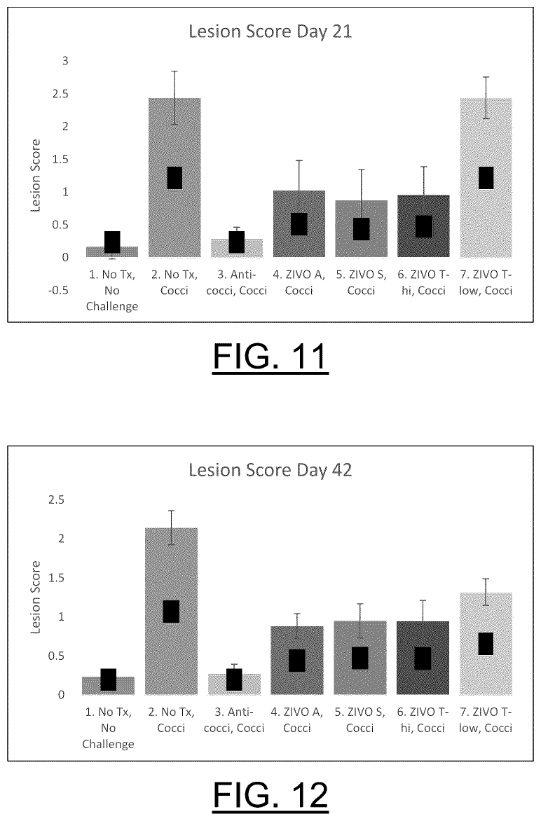

Lesion Scoring—Gross necropsy and lesion scoring were performed on Days 21 and 42. Birds were selected, sacrificed, weighed, and examined for the presence and degree of coccidia lesions and the amount of intestinal gut lining sluffing. CECA damage scores were assessed and recorded as illustrated in FIGS. 11 and 12. By Day 42, the lesion score was significantly reduced across all groups treated with ZIVO A, ZIVO S, ZIVO T-hi, and ZIVO T-low.

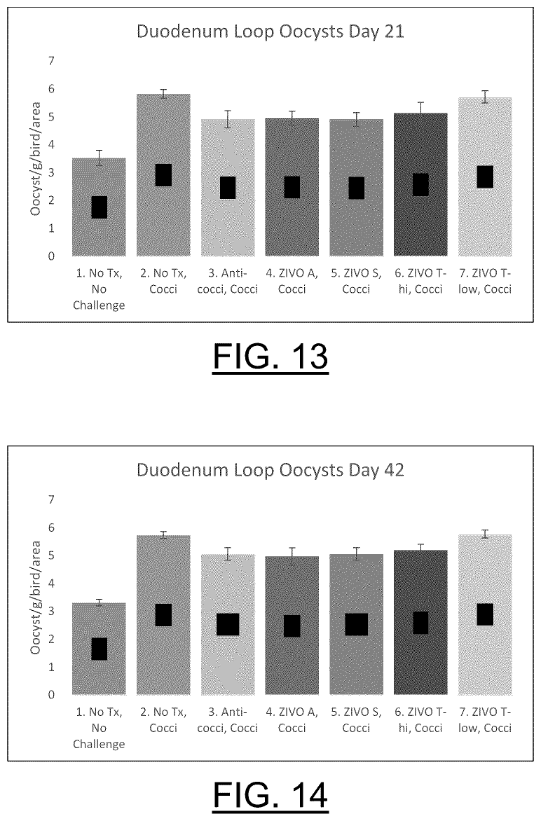

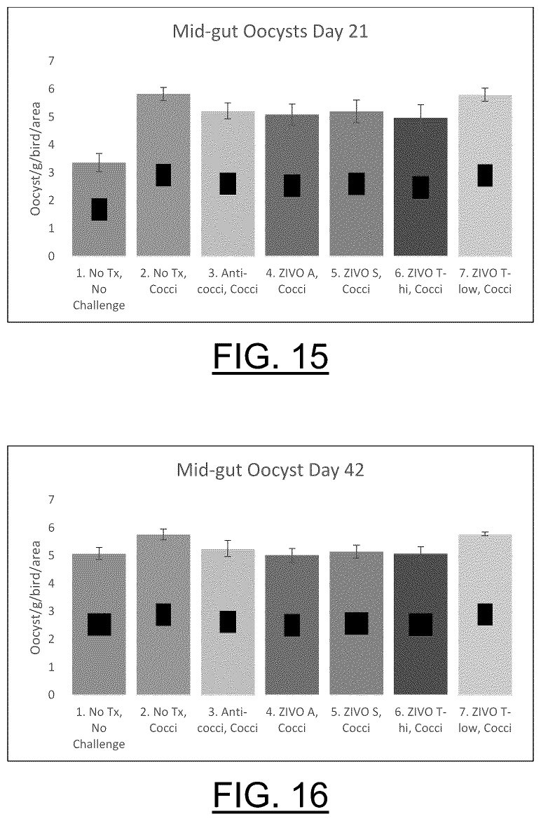

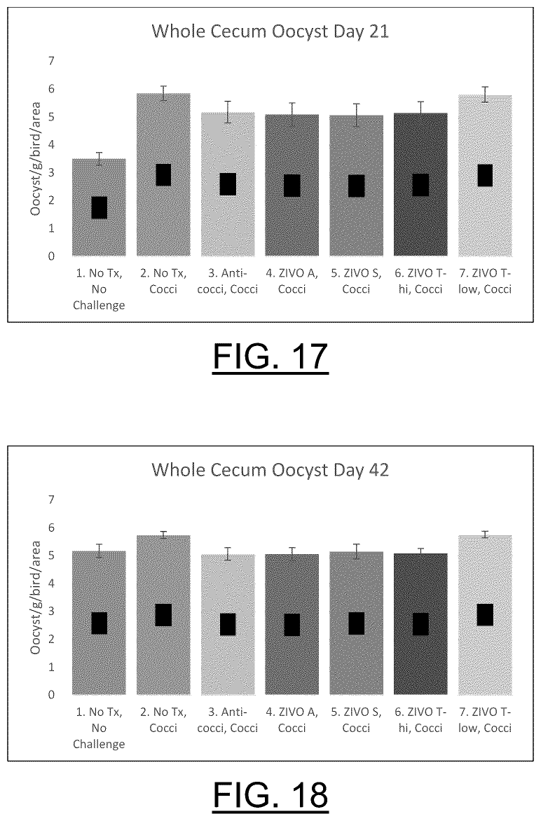

Oocyst Score—Gross necropsy and oocyst scoring were performed on Days 21 and 42 at different locations on the animal. Previously oocyst-inoculated birds were selected, sacrificed, weighed, and examined for the presence and degree of oocysts in their duodenum loop, mid-gut, and whole cecum. The results of the study are illustrated in FIGS. 13 through 18. With respect to the duodenum loop oocyst counts for Days 21 and 42 of FIGS. 13 and 14 respectively, by Day 42 the duodenum loop oocyst count remained relatively unchanged across the groups treated with ZIVO A, ZIVO S, ZIVO T-hi, and ZIVO T-low. The mid-gut oocyst demonstrated almost no change as illustrated in FIGS. 15 and 16 with a similar result for the whole cecum counts as illustrated in FIGS. 17 and 18.

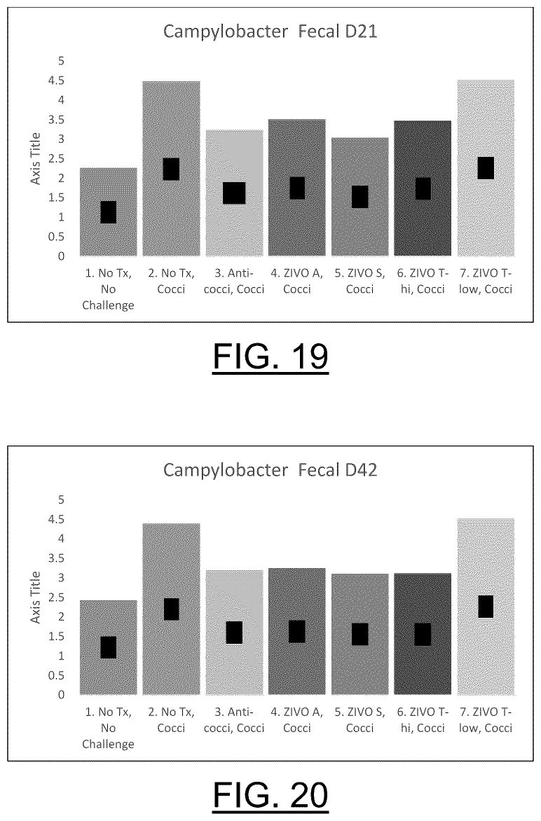

Bacteria—As noted above, coccidiosis damages the gut of the animal, thus often acting as a predisposing factor to the rapid onset of bacterial infection and consequential disease, such as necrotic enteritis. Poultry are susceptible to various bacteria, including Campylobacter, Salmonella, C. perfringens, and E. coli. As illustrated in FIGS. 19 through 30, samples from the cecum as well as from feces were evaluated for the presence of bacteria on both Day 21 and Day 42. The intestinal and fecal samples were analyzed to determine a total aerobic plate count (APC).

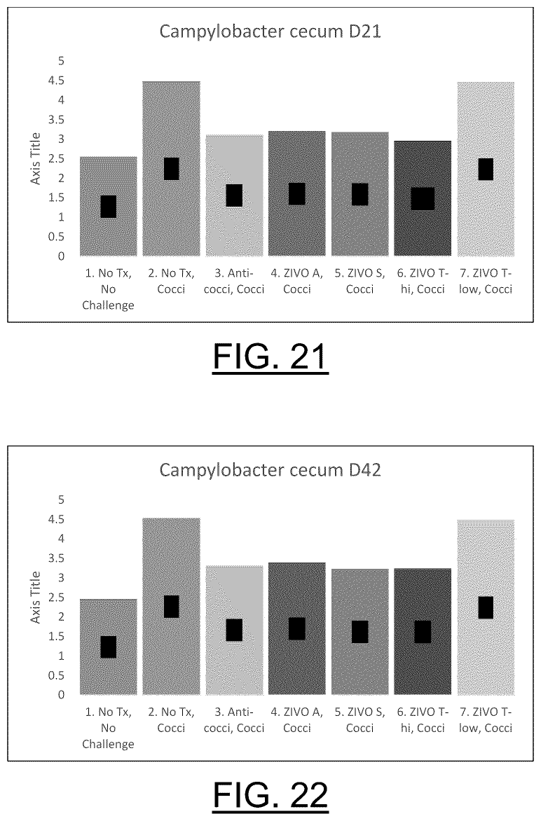

With respect to data related to Campylobacter, FIGS. 19 and 20 illustrate the differences between Day 21 and 42 in which it can be seen that the fecal count generally dropped in all animals treated with ZIVO A, ZIVO S, ZIVO T-hi, and ZIVO T-low. The same is generally true with respect to the results of the Campylobacter cecum count illustrated in FIGS. 21 and 22.

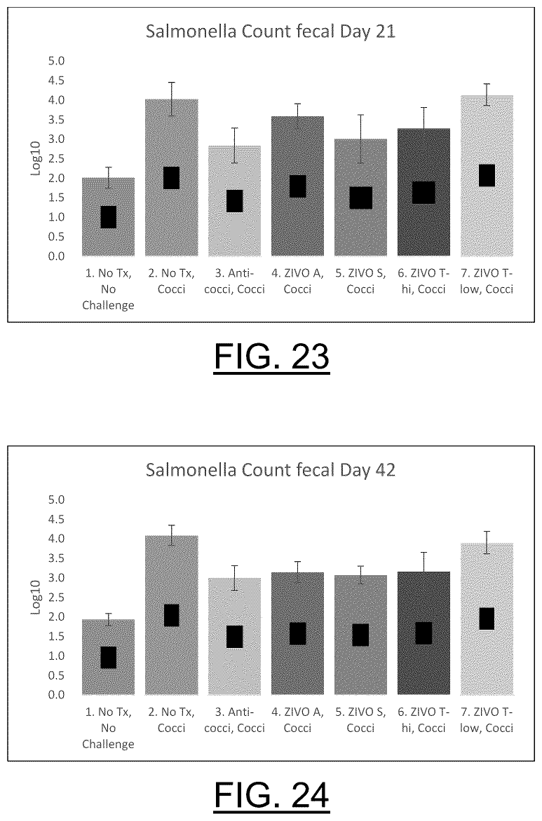

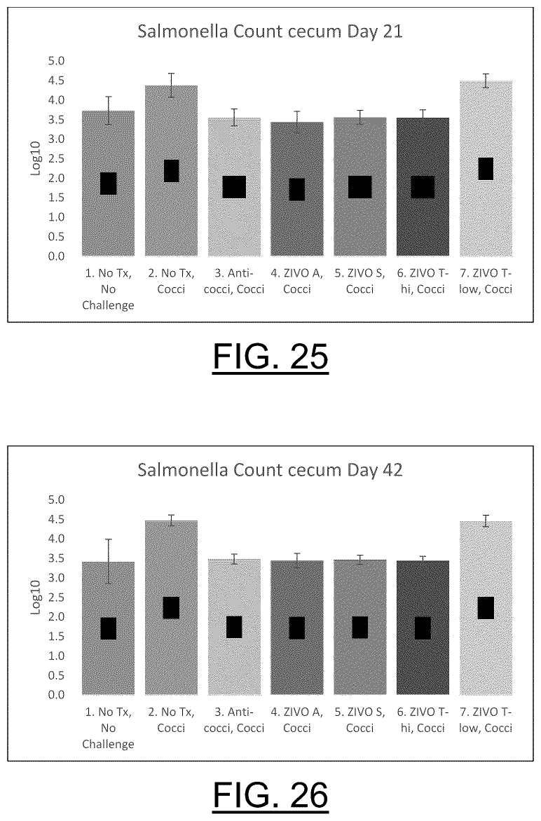

With respect to data related to Salmonella, FIGS. 23 and 24 illustrate the differences between Day 21 and 42 in which it can be seen that the fecal count generally dropped in all animals treated with ZIVO A, ZIVO S, ZIVO T-hi, and ZIVO T-low. The same results generally held true with respect to the results of the Salmonella cecum count illustrated in FIGS. 25 and 26.

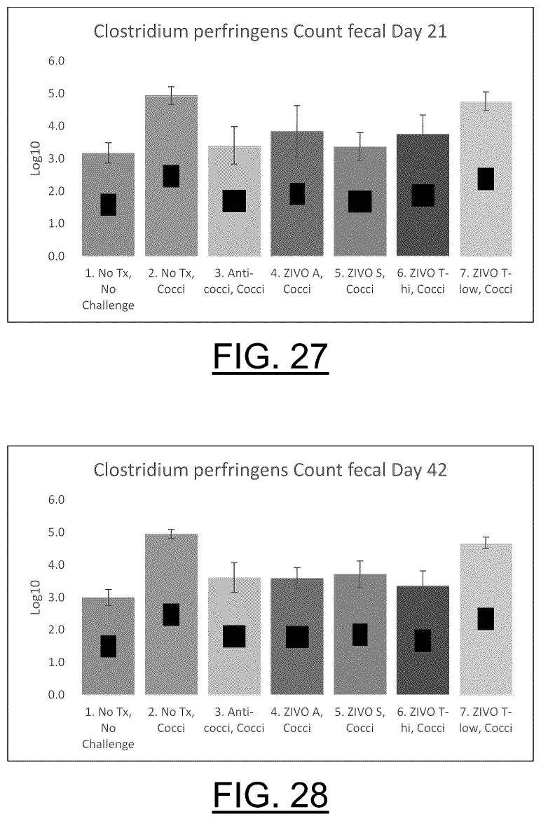

With respect to data related to C. perfringens, FIGS. 27 and 28 illustrate the differences between Day 21 and 42 in which it can be seen that the fecal count generally dropped in all animals treated with ZIVO A, ZIVO T-hi, and ZIVO T-low.

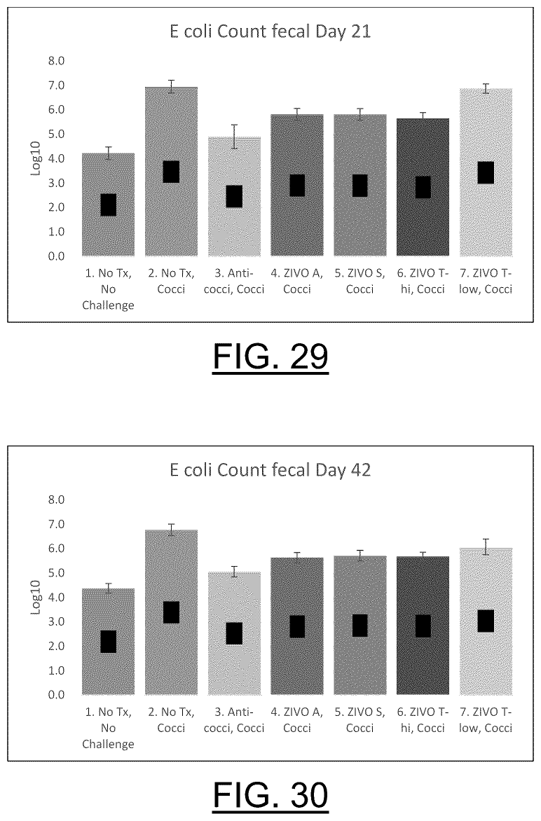

With respect to data related to E. coli, FIGS. 29 and 30 illustrate the differences between Day 21 and 42 in which it can be seen that the fecal count generally dropped in all animals treated with ZIVO A, ZIVO S, and ZIVO T-hi but showed less effect in animals treated with ZIVO T-low.

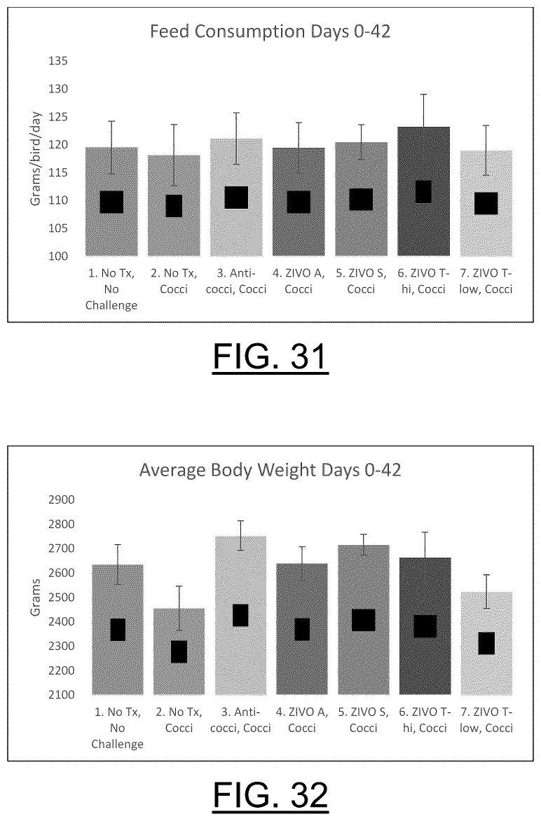

Live Performance Evaluation—Live performance parameters were recorded weekly throughout the study. As illustrated in FIGS. 31-33, the disease challenge environment (cocci-challenge+built-up litter) was employed effectively, as evidenced by the fact that the groups treated with ZIVO A, ZIVO S, ZIVO T-hi, and ZIVO T-low outperformed the untreated and coccidiosis-diseased group for weight gain, feed efficiency, and mortality across all age ranges.

Feed Consumption—As illustrated in FIG. 31, feed consumption was consistently improved in the groups treated with ZIVO A, ZIVO S, ZIVO T-hi, and ZIVO T-low compared with the untreated and coccidiosis-diseased group.

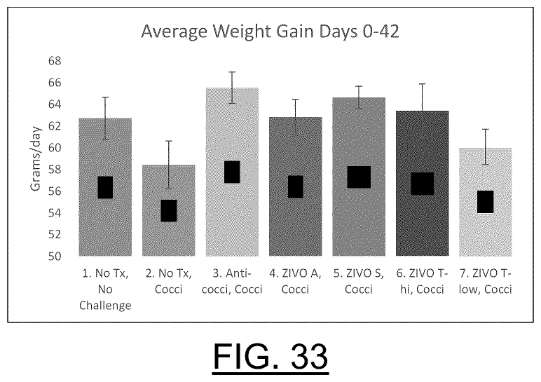

Body Weight Evaluation—As illustrated in FIGS. 32 and 33, individual weights were recorded on for Days 0-42 of the study in both grams and in grams/day respectively. Across all age periods, the average body weight and average body weight gain by groups treated with ZIVO A, ZIVO S, ZIVO T-hi, and ZIVO T-low was significantly increased compared to the untreated and coccidiosis-diseased group.

Results

In general, analysis of the results=supports the conclusion that use of the innovative compound in the treatment of coccidiosis-challenged poultry results in a significant improvement in the health of diseased poultry when compared with untreated poultry. The positive results noted below were identified in the different bacterial variations of the composition of the disclosed inventive concept.

The results are summarized as follows:

FCR showed improvement in the sample poultry treated with the disclosed composition compared with untreated disease-challenged birds.

Mortality was dramatically reduced after Days 0 to 7 in the sample poultry treated with the disclosed composition compared with untreated disease-challenged birds. The level of mortality generally stayed low throughout the study period.

Upon examination of sacrificed sample birds, it was found that the average lesion scores of both the duodenum and the ceca of sample poultry treated with the disclosed composition were lower than the scores of sacrificed untreated disease-challenged birds.

Upon examination of sacrificed sample birds, it was found that the average oocyst count of the duodenum, mid-gut, and cecum of sample poultry treated with the disclosed composition were lower than the scores of sacrificed untreated disease-challenged birds.

It was found that the presence of various bacteria, including Campylobacter, Salmonella, C. perfringens, and E. coli, was generally reduced in treated birds compared with untreated birds.

Average body weight of sample poultry treated with the disclosed composition as greater than the average body weight of untreated disease-challenged birds.

The improvement of the overall health of disease-challenged poultry as a result of treatment with the disclosed inventive composition was achieved without the use of antibiotics.

Overall the inventive composition demonstrates a cost-effective and practical approach to the treatment of disease states in animals.

What is claimed is:

11. The composition of claim 7 wherein said Gram-negative bacteria is a member of the group Variovorax.

21. A composition for the treatment of coccidiosis in animals, the composition comprising effective amounts of a feed ingredient including a lipopolysaccharide derived from Gram-negative bacteria, the composition being deposited under ATCC Item No. SD-8636.

22. The composition of claim 21 wherein said Gram-negative bacteria is a member of the group Rhodobacter.

23. The composition of claim 22 wherein said member of the group Rhodobacter is Rhodobacter sphaeroides.

24. The composition of claim 21 wherein said Gram-negative bacteria is a member of the group Variovorax.

25. The composition of claim 24 wherein said member of the group Variovorax is Variovorax paradoxus.

26. The composition of claim 21 wherein the composition is for the treatment of coccidiosis in poultry.

27. A composition for the modulation of the TLR pathway in animals, the composition comprising a lipopolysaccharide derived from Gram-negative bacteria, the composition being deposited under ATCC Item No. SD-8636.

28. The composition of claim 27 wherein said Gram-negative bacteria-derived lipopolysaccharide is an agonist of the TLR pathway.

29. The composition of claim 27 wherein said Gram-negative bacteria is a member of the group Rhodobacter.

30. The composition of claim 30 wherein said member of the group Rhodobacter is Rhodobacter sphaeroides.

31. The composition of claim 27 wherein said Gram-negative bacteria is a member of the group Variovorax.

32. The composition of claim 31 wherein said member of the group Variovorax is Variovorax paradoxus.

33. A method for treating an animal for subclinical or clinical coccidiosis through modulation of the TLR pathway, the method comprising administering a biomass-based composition including a compound derived from a Gram-negative bacteria at amounts efficacious for the treatment of the subclinical or clinical coccidiosis, the composition being deposited under ATCC Item No. SD-8636.

34. The method for treating an animal of claim 33 wherein said biomass-based composition is administered at the concentration range of between about 0.5 lbs. per ton and about 11.0 lbs. per ton of finished feed.

35. The method for treating an animal of claim 33 wherein said biomass-based composition is administered at the concentration range of between about 3.5 lbs. per ton of finished feed.

36. A method for treating an animal for subclinical or clinical coccidiosis through modulation of the TLR pathway, the method comprising administering a composition including a purified a lipopolysaccharide derived from Gram-negative bacteria at amounts efficacious for the treatment of the subclinical or clinical coccidiosis, the composition being deposited under ATCC Item No. SD-8636.

37. The method for treating an animal of claim 36 wherein said composition is administered at the concentration of about 2.0 mcg and about 20.0 mcg per liter of drinking water.

38. A selective modulator of the TLR pathway comprising a lipopolysaccharide composition derived from a member Gram-negative bacteria, said Gram-negative bacteria derived lipopolysaccharide being an agonist of the TLR pathway, the composition being deposited under ATCC Item No. SD-8636.

39. The selective modulator of claim 38, wherein said Gram-negative bacteria is a member of the group Rhodobacter.

40. The selective modulator of claim 38, wherein said Gram-negative bacteria is a member of the group Variovorax.
